(19) United States
(12) Patent Application Publication
(10) Pub. No.: US 2026/0210959 A1
(43) Pub. Date: Jul. 23, 2026

(54) HIGH DENSITY MICROARRAYS AND USES THEREOF

(71) Applicant: The Regents of the University of Michigan, Ann Arbor, MI (US)

(72) Inventors: Katsuo Kurabayashi, Ann Arbor, MI (US); Shiuan-Haur Su, Ann Arbor, MI (US); Yujing Song, Ann Arbor, MI (US); Andrew Stephens, Ann Arbor, MI (US)

(21) Appl. No.: 19/139,920

(22) PCT Filed: Dec. 18, 2023
(86) PCT No.: PCT/US2023/084641

§ 371 Date: Jun. 17, 2025

Related U.S. Application Data
(60) Provisional application No. 63/433,911, filed on Dec. 20, 2022.

(51) Int. Cl. G01N 33/543; G01N 33/552

(52) U.S. Cl. CPC G01N 33/54393; G01N 33/54386; G01N 33/552

(57) ABSTRACT

The present disclosure relates to high density microarrays, methods of manufacturing the arrays, and uses thereof. In particular, the present disclosure provides high density microarrays of biological molecules that allow for specific and sensitive biological assays.

### STATEMENT OF RELATED APPLICATIONS

This application claims the benefit of U.S. Provisional Patent Application No. 63/433,911, filed Dec. 20, 2022, the contents of which is incorporated by reference herein.

### STATEMENT REGARDING FEDERALLY SPONSORED RESEARCH OR DEVELOPMENT

This invention was made with government support under grant CBET1931905 awarded by the National Science Foundation. The government has certain rights in the invention.

### FIELD

The present disclosure relates to high density microarrays, methods of manufacturing the arrays, and uses thereof. In particular, the present disclosure provides high density microarrays of biological molecules that allow for specific and sensitive biological assays.

### BACKGROUND

Unlocking the potential of protein biomarkers in blood and other bodily fluids is important for the diagnosis, prognosis, and treatment of many diseases including cancer (Borrebaeck, 2018) neurological disorders (Zetterberg and Blennow, 2020), immune dysfunction (Sarma et al., 2020), and cardiac injury (Park et al., 2017). However, current analytical techniques are technologically limited in identifying reliable protein biomarkers in the vast human proteome (Waury et al., 2022). Biomarker discovery typically begins with untargeted approaches, commonly mass spectrometry or transcriptomic analysis, generating large lists of potential candidates related to the disease state of interest. However, these approaches require a large amount of sample input for accurate analysis of low-abundance proteins and involve relatively complex and expensive processes. The data analysis is also time-consuming and contributes to low-throughput screening. As such, these candidate biomarkers need to be further verified using multiplex immunoassays, which provide quantitative analysis and adequate sample throughput to meet requirements for statistical significance (Rifai et al., 2006). Increasingly, this validation step is requiring more sensitive measurement of low-abundance biomarkers, which conventional Luminex xMAP and antibody microarrays have difficulty achieving due to lower sensitivity compared to the gold-standard single-plex enzyme linked immunosorbent assay (ELISA). Repeatability and standardization between laboratories are concerns as well (Tighe et al., 2013; Ellington et al., 2010).

To address this gap, numerous technologies have emerged for the sensitive and multiplex detection of proteins (Cohen and Walt, 2019; Ren et al., 2021), with single-molecule counting “digital ELISA” demonstrating up to 1000-fold improvement in sensitivity over conventional ELISA (Duffy, 2023). Digital ELISA is achieved by the isolation and detection of single protein molecules on antibody-coated microbeads confined in fL-nL microwells or droplets. Measuring multiple analytes in a single run (multiplexing) is commonly enabled by dye-encoded microbeads (Rissin et al., 2013). Due to the binary nature of digital ELISA, Poisson statistical theory dictates that a minimum number of microbeads must be analyzed to ensure an acceptable theoretical noise level per analyte (Zhang and Noji, 2017). A common approach to signal readout in digital ELISA is to load a suspension of microbeads into microwell arrays by gravity, but this stochastic process can leave more than 30-50% of microwells empty. Multiplexing in digital ELISA therefore requires more beads to be analyzed in an individual microwell array and is limited by this gravity-loading process. Accordingly, increasing the bead loading efficiency has been an active area of research. Improving on the original optical fiber-based digital ELISA by Rissin et al. (2010), Noji and coworkers demonstrated that the background signal of a digital bioassay can be reduced by interrogating a large number of microwells (Kim et al., 2012). The Lammertyn group has shown that bead loading efficiency can be improved by using digital microfluidics to actuate a droplet containing suspended magnetic beads back and forth over hydrophilic microwells while a magnet pulls beads into the microwells (Witters et al., 2013). More recently, they have demonstrated improved loading efficiency using passive hydrophobic surfaces with hydrophilic microwells fabricated using scalable methods (Tripodi et al., 2018; Zandi Shafagh et al., 2019). Quanterix Corp. has demonstrated improved assay performance by tuning the total number of beads used in the reaction combined with a magnetic-meniscus sweeping strategy to assist bead loading (Kan et al., 2020). A hybrid spatial-spectral multiplex encoding strategy by pre-seeding the microwell arrays with beads rather than mixing them with the sample solution, allowing multiplexing for up to 14 cytokines is described in Song et al. (Song et al., 2021a; 2021b; 2021c). Despite these improvements, the total multiplex capacity for bead-based digital ELISA remains limited spectrally by the finite number of flurophores available to encode beads, and spatially by the fixed size of microwell arrays.

What is needed are immunoassays that provide high multiplex capacity and allow for small sample volumes.

### SUMMARY

The present disclosure relates to high density microarrays, methods of manufacturing the arrays, and uses thereof. In particular, the present disclosure provides high density microarrays of biological molecules that allow for specific and sensitive biological assays.

In some exemplary, non-limiting embodiments, provided herein is an integrative platform for the simultaneous measurement of a large number of analytes in small volumes of biological fluids. The platform is a microarray-format ELISA assay that uses the principle of single-molecule counting for signal amplification. Protein patterning based on photolithography is used to achieve single molecule-counting by sub-dividing addressable arrays of capture molecule (e.g., antibody) spots into hundreds of thousands of individual microwells. Arrays of microwells can be readily integrated into several microfluidics biosensing applications. The systems provide performance benefits to limit of detection (LoD), lower limit of quantification (LLoQ), measurement precision, and total multiplex capacity that the platform provides. The utility of the platform was demonstrated through longitudinal serum measurement of six cytokines in response to glioma tumor progression in a cohort of mice.

For example, in some embodiments, provided herein is a method of arraying biological molecules on a substrate, comprising: one or more or all of the steps of a) patterning the substrate with protein-binding silane spots; b) applying an antibiofouling coating to the substrate; c) coating the substate with the biological molecules; and d) contacting the substrate with an exfoliation material to remove nonsilane-bound protein. In some embodiments, the substrate is a surface. In some embodiments, the substrate or surface comprises a material selected from, for example, glass, plastic, or metal. In some exemplary embodiments, the substrate is selected from, for example, a well in a microtiter plate, a film, a microscope slide, or a bead. In some embodiments, the substrate comprises lithographically patterned microwells.

The present disclosure is not limited to particular exfoliation materials. Examples include but are not limited to, tape (e.g., a low residue tape such as low-residue semiconductor grade tape or cellophane tape). In some embodiments, the antibiofouling coating comprising polysiloxanes (e.g., hydroxy-terminated polydimethylsiloxane).

The present disclosure is not limited to particular biological molecules. Examples include but are not limited to, proteins (e.g., antibodies), nucleic acid, lipids, or carbohydrates. In some embodiments, each of the spots comprises one or more distinct biological molecules. In some embodiments, the array comprises the same or different biological molecules. In some embodiments, each of the spots is less than 2 μm in diameter (e.g., less than 1 μm, less than 750 nm, less than 500 nm, less than 250 nm, less than 100 nm, less than 50 nm, less than 25 nm, less than 10 nm or less than 1 nm) in diameter. In some embodiments, the biological molecules are only present in the spots.

Also provided is a substrate comprising an array of biological molecules manufactured by a method described herein.

Additional embodiments provide a kit or system comprising a substrate described herein. In some embodiments, the kit or system further comprises a microfluidic device, for example, in some embodiments, the substrate is integrated into the microfluidic device.

Further provided is a method of performing an assay, comprising: a) contacting a substrate described herein with a test sample; and b) measuring one or more properties of the biological molecules in the presence and absence of the test sample. In some embodiments, the assay measures the presence and/or amount of an analyte in the test sample. In some embodiments, the assay is an ELISA (e.g., single molecule ELISA). In some embodiments, the ELISA comprises detection in a water in oil droplet. The present disclosure is not limited to a particular analyte. Examples include but are not limited to a protein, small molecule, lipid, or nucleic acid.

Additional embodiments are described herein.

### BRIEF DESCRIPTION OF THE DRAWINGS

FIG. 1. Digital Protein Microarray (A) Conventional protein microarray schematic. (B) Schematic of the conventional protein microarray detection approach. (C) DPMA device fabricated on a standard 75×25 mm form factor glass substrate. (D) Schematic of single-molecule detection approach used in DPMA. (E) Antibody-coated microwell structures in DPMA. Left: Brightfield microscope image of microwell array sensors at 5× magnification (top) and 60× magnification showing individual microwells (bottom). Right: Composite fluorescence image demonstrating multiplex functionalization of microwell arrays. Bottom: Green fluorescence channel image of microwells shown in bottom-right. Antibody-coated microwells appear bright green while uncoated regions appear dark. (F) Assay process flow of digital ELISA in DPMA. Panel 1: Sample containing target protein (yellow circles) is mixed with a biotinylated detection antibody (purple) in the flow cell over microwell array sensors (gray, bottom). Panel 2: The flow cell is flushed with washing buffer, then a buffer containing streptavidin-HRP is injected into the flow cell to label the antibody-analyte immunocomplexes. Panel 3: The flow cell is washed again, then the HRP substrate is injected into the flow cell (blue), followed by HFE oil (light yellow) to form water-in-oil droplets. Panel 4: DPMA chips are imaged using epifluorescence microscopy and an automated X-Y-Z stage. (G) Representative image of microwell array sensor readout. Bar chart shown is representative of data from a 3-plex measurement.

FIG. 2. Fabrication and antibody functionalization of DPMA. (A) Cross-sectional microfabrication diagram (top-to-bottom) of microwell arrays. Step 1: Lithographic patterning of microwell arrays in positive photoresist. Step 2: Dry etching of microwells into a fused silica wafer. Step 3: Silanization of APTES within microwells using chemical vapor deposition. The chemical structure and orientation of APTES is shown in the circular inset figure. Step 4: Photoresist is removed and Rain-X™ is applied to create a hydrophobic surface outside of the microwells. (B) Hydrophilic-in-hydrophobic microwell array surface after microfabrication steps have been completed. Left: Scanning electron microscope (SEM) image taken at 45° perspective of microwell array. Right: Water contact angle measurement of APTES (top) and Rain-X™ (bottom) treated regions of a glass substrate. (C) Verification of water-in-oil droplet formation. (D) Process flow of antibody functionalization (left-to-right). Step 1: Antibodies in coating buffer are dispensed on microwell array surfaces. Step 2: Antibody solution is allowed to incubate and dry. Step 3: Nonspecifically adsorbed antibody on the surface outside of the microwells is removed using the tape exfoliation method. Step 4: Antibody-functionalized devices are ready for use. (E) Assay schematic used to visualize capture antibody coating and verify specificity of patterning within microwells. (F) Microwell array before (left) and after (right) tape exfoliation with coating visualized by labeling with AF448 goat anti-rat IgG antibody. Detail is shown in inset figures, scale bars are 20 μm

FIG. 3. Photolithographic protein patterning enables miniaturization of digital ELISA. (A) Confocal fluorescence microscope images of microwell arrays of 16, 8, and 4 μm well-to-well pitch (left to right). Top: maximum intensity z-stack projection. Bottom: Z-stack profile taken from a representative cross section, enlarged to show detail. (B-C) Standard curves for microwell arrays of well-to-well pitch 4-20 μm. Each data point is the average of 3 measurements, error bars are one standard of deviation above or below the mean. (D) Resolution of molecular concentration (RMC) versus concentration for 4 μm, 20 μm, and commercial ELISA and Luminex mouse IL-6 kits. The parameter u indicates the fold difference of analyte concentration that could be resolved with 90% confidence. (E) LoD and LLoQ as total sensor area is increased. (F) Maximum possible sensors versus substrate area for 4 (blue), 8 (red), and 16 (black) μm microwell pitches benchmarked against commercial microarray technologies (empty circles). Dotted lines indicate substrate sizes relevant to various life sciences applications.

FIG. 4. Application of DPMA in glioma mouse model serum biomarker analysis. (A) Experimental design. (B) Example in-vivo imaging system (IVIS) images showing bioluminescent tumors in mice bearing no tumor, mid-stage, and late-stage tumors (indicated by the red arrows). (C) Schematic of DPMA used in this study. (D) Serum biomarker measurements by DPMA. Each data point corresponds to the serum level of an individual mouse in the wildtype or mutant group. Bars are the group mean, error bars are one standard deviation of the group mean, and significance is calculated from group mean. *P<0.05, **P<0.005, ***P<0.001. ANOVA. Measurements below the assay LoD were excluded from analysis. Data points below LoD for TNF-α are plotted at 0 μg/mL.

FIG. 5. DPMA fluorescence imaging setup. (A) DPMA chip in standard 75×25 mm microscope slide nest. (B) Programable X-Y-Z Motorized Stage (Prior Scientific). (C) CMOS camera for image acquisition (XIMEA xiC). (D) Laptop for LabView and image acquisition control. (E) Fluorescence light source (Nikon). (F) Inverted epifluorescence microscope (Nikon Eclipse Ti) with 10× lens (Nikon).

FIG. 6. Microfabrication cross sectional diagram of all steps involved with DPMA fabrication.

FIG. 7. Verification of tape exfoliation method. Fluorescence image of a piece of low-residue tape that had been used for tape exfoliation.

FIG. 8. 6-plex standard curve for the mouse glioma biomarker panel used FIG. 4 of the manuscript.

FIG. 9. Cross-reactivity assessment of the 6-plex mouse glioma biomarker panel.

FIG. 10. DPMA correlation to ELISA for mouse IL-6. Each data point was measured in with mouse serum with animals known to have a relatively low, mid-range, or high serum concentration of IL-6.

FIG. 11. Microwell dimensions as measured by (A) scanning electron microscope (SEM) (Hitachi SU8000) cross section and (B) white light interferometry (WLI) (Zygo NewView 5000).

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

“Biomolecule,” as used herein, includes large macromolecules (or polyanions) such as proteins, carbohydrates, lipids, and nucleic acids, as well as small molecules such as primary metabolites, secondary metabolites, and nucleotides.

“Biomarker,” as used herein, refers to any substance in which its presence, absence, or relative quantity may indicate a particular disease state in a subject. The biomarker includes, but is not limited to, proteins, polypeptides, nucleic acids, small molecules and the like.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Such examples are not however to be construed as limiting the sample types applicable to the present disclosure.

“Biological sample,” as used herein, includes biological fluids, including, but not limited to, whole blood, serum, plasma, synovial fluid, cerebrospinal fluid, bronchial lavage, ascites fluid, bone marrow aspirate, pleural effusion, urine, as well as tumor tissue or any other bodily constituent or any tissue culture supernatant that could contain a molecule of interest.

“Isolating,” as used herein, means any process which results in each individual component of a mixture, such as a single capture agent, or a single capture agent-biomolecule-detection agent complex, being isolated such that only one component is in any one location; that location being optically distinct from any other location. The isolation can be accomplished by utilizing a solid support, as described herein.

“Labeling agent,” as used herein, refers to any molecule or compound which facilitates detection by reacting with the detection moiety to produce a detectable reaction product.

“Magnetic bead,” as used herein, refers to so-called magnetic beads, magnetic microbeads, paramagnetic particles, magnetically attractable particles, magnetic spheres, and magnetically responsive particles. These terms are often used interchangeably throughout the field. As such, “magnetic beads” include any of the particles capable of being manipulated in a liquid with the application of a magnetic field. The magnetism of the bead may include paramagnetic, superparamagnetic, ferromagnetic, antiferromagnetic, and ferrimagnetic properties.

“Polynucleotide” or “oligonucleotide” or “nucleic acid,” as used herein, means at least two nucleotides covalently linked together. The polynucleotide may be DNA, both genomic and cDNA, RNA, or a hybrid, where the polynucleotide may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. Polynucleotides may be single- or double-stranded or may contain portions of both double stranded and single stranded sequence. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The proteins may be modified by the addition of sugars, lipids or other moieties not included in the amino acid chain. The terms “polypeptide”, “protein,” and “peptide” are used interchangeably herein.

“Probe,” as used herein, refers to a molecule that binds specifically or selectively to a molecule. The probe may be a nucleic acid, an aptamer, an avimer, receptor-binding ligands, binding peptides, protein, small organic molecules, or a metal ligand. The probe may be an antibody, antibody fragment, a bispecific antibody or other antibody-based molecule or compound designed to bind to a specific biomolecule. The probe may be the same type of molecule as the biomolecule, for example, a protein biomolecule may be bound by a peptide-based probe. Single stranded polynucleotides of complementary sequence may hybridize to form double stranded polynucleotides. The probe may be a different type of molecule from the biomolecule, for example, a polynucleotide probe may bind to a protein biomolecule.

“Separating,” as used herein, means any spatial partitioning of one or more components from the remainder. Separation therefore includes, but is not limited to, fractionation as well as to a specific and selective enrichment, depletion, concentration and/or isolation of certain fractions or analytes contained in a sample.

“Solid support,” as used herein, refers any solid device capable of isolating individual components of a mixture. For example, the solid support may an array with a spatially defined areas in which individual components are isolated by a surface treatment or a magnetized layer. The solid support may have distinct structures (e.g., chambers, sections, wells, or channels) which separate the components, for example, a microfluidic device or a microtiter plate.

### DETAILED DESCRIPTION

The present disclosure relates to high density microarrays, methods of manufacturing the arrays, and uses thereof. In particular, the present disclosure provides high density microarrays of biological molecules that allow for specific and sensitive biological assays.

Described herein is a microarray-format digital ELISA platform called digital protein microarray (DPMA) that achieves sensitive, multiplex, and high-resolution measurements using minimal sample volume. The entirely spatially-encoded multiplex strategy of DPMA allows for the highest potential multiplex capacity digital ELISA assay to date. Without needing to consider optical cross-talk due to overlap of fluorophores, the multiplex capacity of DPMA is increased. The sensor size can be miniaturized using the photolithographic protein patterning methods described herein. The photolithography-based protein patterning enables precise control of microwell and microwell array size, and total microarray number. As microwell density is increased, key sensor performance metrics of limit of detection (LoD), lower limit of quantification (LLoQ), and resolution of molecular concentration improve with no trade-off in performance metrics at the microwell array sizes and densities investigated. The DPMA technology described herein finds use, for example, as a platform technology that can be used in high-performance, high-multiplex proteomics studies, as in the application to mouse serum analysis demonstrated herein or integrated into other microfluidics applications requiring sensitive detection of protein analytes.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

As described herein, embodiments of the present disclosure provide high density microarrays with single molecule detection capacity and low limits of detection, and methods of generating such arrays. In some exemplary embodiments, the arrays are made by a method, comprising: a) patterning the substrate with protein-binding silane spots; b) applying an antibiofouling coating to the substrate; c) coating the substate with the biological molecules; and d) contacting the substrate with an exfoliation material to remove nonsilane-bound protein.

The present disclosure is not limited to particular exfoliation materials. Examples include but are not limited to, tape (e.g., a low residue tape such as low-residue semiconductor grade tape or cellophane tape). In some embodiments, the antibiofouling coating comprising polysiloxanes (e.g., hydroxy-terminated polydimethylsiloxane).

The present disclosure is not limited to particular biological molecules for inclusion on the array as capture agents or detection agents. Examples include but are not limited to, proteins (e.g., antibodies), nucleic acid, lipids, or carbohydrates. In some embodiments, each of the spots comprises one or more distinct biological molecules. In some embodiments, the array comprises the same or different biological molecules. In some embodiments, each of the spots is less than 2 μm in diameter (e.g., less than 1 μm, less than 750 nm, less than 500 nm, less than 250 nm, less than 100 nm, less than 50 nm, less than 25 nm, less than 10 nm or less than 1 nm) in diameter. In some embodiments, the biological molecules are only present in the spots.

In some embodiments, the substrate is a surface. In some embodiments, the substrate or surface comprises a material selected from, for example, glass, plastic, or metal. In some exemplary embodiments, the substrate is selected from, for example, a well in a microtiter plate, a film, a microscope slide, or a bead. In some embodiments, the substrate comprises lithographically patterned microwells.

The solid support may be smooth, having a substantially planar surface, or it may contain a variety of structures such as wells, grooves, depressions, channels, elevations, chambers, or the like, in which each capture agent or capture agent-molecule-detection agent complex is isolated. The solid support may be a microfluidic device comprising a series of microchannels which isolate the individual capture agents or capture agent-molecule-detection agent complexes. The solid support may be a multi-well plate comprising a vast number of wells which isolate the individual capture agents or capture agent-molecule-detection agent complexes. The solid surface may be a magnetic array such that individual regions of magnetism result in the isolation of each capture agent or capture agent-molecule-detection agent complex on a substantially planar surface. In some embodiments, the solid support is a microplate or microfluidic device.

The solid support may be composed of any of a wide variety of materials, for example, polymers, plastics, resins, polysaccharides, silica or silica-based materials, carbon, metals, inorganic glasses, membranes, or any combinations thereof. The solid support material may be treated, coated, modified, printed or derivatized using polymers, chemicals to impart desired properties or functionalities to the array support surface. Preferred solid support material may be compatible with the range of conditions encountered during the assay including salt concentrations and solvents, be stable under the application of magnetic fields, and be optically transparent.

The solid supports may be those commercially available or formed using common methods including, but not limited to, film deposition processes, such as spin coating and chemical vapor deposition, laser fabrication or photolithographic techniques, wet chemical or plasma etching methods, and/or molding or casting.

The solid support may be sealed prior to detection to prevent evaporation or migration of the contents in the individual locations during the remainder of the analysis. The solid support may be sealed after the isolation or after the optional addition of a labeling agent. Sealing methods include, for example, overcoating the top surface of the solid support with a sealing fluid (e.g., a non-aqueous fluid, such as oil) or using a sealing tape to isolate each individual location.

In some embodiments, the present disclosure provides a method of performing an assay (e.g., to detect the presence of an analyte) using the substrates described herein. In some embodiments, the assay is an ELISA (e.g., single molecule ELISA). In some embodiments, the ELISA comprises detection in a water in oil droplet. The present disclosure is not limited to a particular analyte. Examples include but are not limited to a protein, small molecule, lipid, or nucleic acid.

Determining the presence of absence of the detection agent may be done directly or indirectly. In the case of direct detection, the detection agent may comprise a detection moiety that may be directly measured. For example, if the detection moiety includes a dye or radioactive isotope, presence of the detection agent may be determined with optical detection of the dye, either fluorescent or visible detection, or infrared spectroscopy or autoradiography, respectively. In the case of indirect detection, the detection agent may comprise a detection moiety that reacts with a labeling agent to form a detectable reaction product.

In some embodiments, the detection agent comprises a detection moiety which can be directly detected. In some embodiments, the method may further comprise adding a labeling agent to the separated capture agents and the capture agent-molecule-detection agent complexes, such that the labeling agent reacts with the detection moiety to produce a reaction product. Thus, in some embodiments, determining the presence or absence of the detection agent comprises measurement of the reaction product. The labeling agent may be added before or after isolation of the capture agent/capture agent-molecule-detection agent complexes into individual locations, preferably after isolation.

In some embodiments, the labeling agent is a substrate for an enzyme included in the capture agent such that upon contact with the enzyme converts the labeling agent into a chromogenic, fluorogenic, or chemiluminescent reaction product, which is detectable. In some embodiments, the labeling agent is an enzyme and the substrate is included in the capture agent. Any known chromogenic, fluorogenic, or chemiluminescent labeling agents may be selected for conversion by many different enzymes. In exemplary embodiments, the enzyme may be beta-galactosidase, horseradish peroxidase, or alkaline phosphatase. The substrate can respectively be an eta-galactosidase, horseradish peroxidase, or alkaline phosphatase well known in the art that are labeled or create a measurable signal upon enzymatic reaction, including, but not limited to: 3,3′,5,5′-tetramethylbenzidine, 3,3′-diaminobenzidine, 2,2′-azino-bis(3-ethylbenzothiazoline-6-sulphonic acid), p-nitrophenyl phosphate, 2,2′-azinobis [3-ethylbenzothiazoline-6-sulfonic acid]-diammonium salt, o-phenylenediamine dihydrochloride, or other enhanced fluorescent or chemiluminescent derivatives thereof.

The detection methods and type of detector employed depend on the nature of the capture agent, detection agent, or labeling agent reaction products. Non-limiting examples of detection methods include optical imaging (fluorescence and visible), Raman scattering, spectroscopy (e.g., infrared, atomic, fluorescence or visible spectroscopies), absorbance, circular dichroism, electron microscopies (e.g., scanning electron microscopy (SEM), x-ray photoelectron microscopy (XPS)), light scattering, optical interferometry and other methods known in the art based on measuring changes in refractive index, diffraction, absorption, and fluorescence technologies.

In some embodiments, the detector may comprise more than one light source and/or a plurality of filters to adjust the wavelength and/or intensity of the light source. In some embodiments, the detector may also include a microscope (light or fluorescent) and/or a camera to capture the detection of the optical output of the detection method. The camera maybe a CCD (charge-coupled device) or CMOS (complementary metal-oxide-semiconductor) camera or similar camera known in the art. By using a camera with an electrical image converter, such as a CCD or CMOS chip, high local resolution can be achieved. The detector may also include a computer or controller used to control the light source, the filters, and/or execute any imaging processing software.

The detector may capture the optical output of the entire solid support at one time. Or the detector may move throughout the solid support during the detection to survey the entire solid support for the presence/absence of capture agents and detection agents.

A measure of the concentration of the molecule may be based on the number and/or fraction of locations determined to contain a capture agent and a detection agent. The concentration may be based on the fraction of locations comprising both the capture agent and the detection agent compared to locations comprising only the capture agent. The concentration may be based on the fraction of locations comprising both the capture agent and the detection agent compared to total locations.

The present disclosure provides systems and/or kits (e.g., reagents, computer software, instruments, etc.) for detecting at least one molecule in a sample. In some embodiments, the systems comprise at least one capture agent comprising and at least one detection agent comprising a second probe configured to bind the one of the at least one molecule.

The capture agent may include different labels or detection chemistries, including for example, fluorescent, chemiluminescent, bioluminescent, or isotopic labels. The detection agent may comprise a second probe configured to bind the same molecule as the capture agent.

The nature of the first and second probes will depend on the type of target molecule. For example, when the target molecule is a protein, the first and second probes may comprise proteins, particularly antibodies or fragments thereof, other proteins, peptides or small molecules. If the target molecule is a nucleic acid, the probes may be a nucleic acid binding protein or a complementary nucleic acid, if the target molecule is a single-stranded nucleic acid. When the target molecule is a carbohydrate, the first and second probes may include, for example, antibodies, lectins, and selectins. Suitable target molecule/probe pairs can include, but are not limited to, antibodies/antigens, receptors/ligands, proteins/nucleic acid, nucleic acids/nucleic acids, enzymes/substrates or inhibitors, carbohydrates (including glycoproteins and glycolipids)/lectins or selectins, proteins/proteins, and proteins/small molecules. In some embodiments, the first probe and the second probe are independently selected from a protein, a peptide, a nucleic acid, a carbohydrate, a small molecule, and a ligand. In exemplary embodiments, the first probe is an antibody. In exemplary embodiments, the second probe is an antibody.

The detection agent may further comprise a detection moiety selected from the group consisting of a dye, a radiolabel, an enzyme, and an enzyme substrate. In some embodiments, the detection moiety is a fluorescent dye. In some embodiments, the detection moiety is an enzyme or enzyme substrate. In certain embodiments, the enzyme is beta-galactosidase, alkaline phosphatase or horseradish peroxidase.

The system or kit may further comprise a labeling agent. The labeling agent reacts with the detection moiety to produce a reaction product.

The system or kit may also comprise a sample (e.g., positive and/or negative control samples), a solid support, a detector, and/or software configured to determine the presence or absence of the capture agent and the detection agent from the output of the detector. In some embodiments, an instrument is provided that automates one or more of the steps of the methods described herein. For example, in some embodiment, the instrument comprises software that controls incubations time to, for example, start and stop reactions such that the pre-equilibrium incubations times described herein are achieved.

In some embodiments, the kit or system further comprises a microfluidic device, for example, in some embodiments, the substrate is integrated into the microfluidic device.

The sample includes any composition which comprises the molecule of interest. The sample may be obtained from any source, including bacteria, protozoa, fungi, viruses, organelles, as well higher organisms such as plants or animals, including humans. Samples can be obtained from other sources, including, but not limited to environmental sources, food products, and forensic samples. In some embodiments, the sample is a biological sample.

The software may be supplied with the systems in any electronic form such as a computer readable device, an internet download, or a web-based portal. The software may be integrated with the detector to not only determine the presence or absence of the capture agent and the detection agent from the output of the detector, and/or a sample but also control the detector components. The software may allow a user to view results in real-time, review results of previous samples, and view reports. The software may output data in the forms of images, graphs, charts, or raw values. The software may also be capable of calculating statistics and making comparisons between data sets.

The systems can also comprise instructions for using the components of the systems. The instructions are relevant materials or methodologies pertaining to the systems. The materials may include any combination of the following: background information, list of components and their availability information (purchase information, etc.), brief or detailed protocols for using the systems, trouble-shooting, references, technical support, and any other related documents. Instructions can be supplied with the systems or as a separate member component, either as a paper form or an electronic form which may be supplied on computer readable memory device or downloaded from an internet website, or as recorded presentation.

The system or kit may further include reagents, computer software, instruments, etc. for obtaining, processing, or preparing a sample. For example, the system may include instruments or devices for taking a sample from a patient (e.g. finger pricks, needles, syringes, and the like), sample separation or pre-processing devices (e.g. plasma separation (apheresis machines), filtration devices, centrifuges, and the like), or extraction or sample stabilizing or separation buffers. In some embodiments, the instruments for obtaining, processing, or preparing a sample may be integrated into any of the devices described above within the system.

Individual member components of the kit or system may be physically packaged together or separately. The kits can also comprise instructions for using the components of the kit. The instructions are relevant materials or methodologies pertaining to the kit. The materials may include any combination of the following: background information, list of components and their availability information (purchase information, etc.), brief or detailed protocols for using the system, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

### EXPERIMENTAL

The following examples are provided in order to demonstrate and further illustrate certain preferred embodiments and aspects of the present invention and are not to be construed as limiting the scope thereof.

### Example 1

### Materials and Methods

### Materials

100 mm fused silica wafers were purchased from University Wafer Inc, Boston, MA, and positive photoresist MEGAPOSIT™ SPR™220 were purchased from Dow Chemical, USA. (3-Aminopropyl)triethoxysilane (APTES) was obtained from Millipore Sigma, St. Louis, MO. Alexa Flour™ 488 (AF488) goat anti-rat IgG, mouse IL-6, and TNF-α capture and biotinylated detection antibody pairs were purchased from BioLegend and LCN2, IFN-Y, CXCL-12, and G-CSF DuoSET ELISA kits from R&D Systems. The Luminex™ kit was purchased from R&D Systems. Avidin-HRP, QuantaRed™ enhanced chemifluorescent HRP substrate, bovine serum albumin (BSA), Casein blocking buffer, and PBS SuperBlock blocking buffer were obtained from Thermo Fisher Scientific. Phosphate buffered saline (PBS) was obtained from Gibco™, Sylgard™ 184 clear polydimethylsiloxane (PDMS) was obtained from Dow Corning, and fluorocarbon HFE oil (Novec™ 7500) was obtained from 3M™.

### Fabrication of Glass Microwell Arrays

Fused silica wafers were first cleaned in piranha solution (3:1 v/v H₂SO₄:H₂O₂) and spin-coated with positive photoresist at 3500 RPM to a thickness of 3 μm. Contact-mode photolithography (KARL SÜSS MA/BA6) was used to define arrays of microwells 2.5 μm in diameter arranged in a hexagonal lattice with center-to-center pitch 4-20 μm, depending on the experiment (FIG. 2A, step 1). Microwell features were then etched into the fused silica wafer to a depth of 3.5 μm using deep glass reactive ion etching (DGRIE, ~2500 Å min etch rate, Advanced Plasma System, SPTS) (FIG. 2A, step 2). DPMA chips were finally cut from the fused silica wafers to a form factor of 75×25 mm using a dicing saw (ADT 7100, Advanced Dicing Technologies, Israel) and stored until use.

### Selective Surface Silanization

With the photoresist in-tact from the microfabrication process, DPMA chips were treated with O2 plasma (50 W, 100% O2, 0.7 Torr, 60 s, Covance, Femto Science, South Korea) to create surface hydroxyl groups inside of the glass microwells. Then, APTES was grafted inside of microwells by chemical vapor deposition (CVD) by placing DPMA chips in a vacuum chamber containing a ~100 μL of liquid APTES at 75° C. for 3 h (FIG. 2A, step 3). DPMA chips were then removed from the vacuum chamber and sonicated in acetone for 5 min and isopropyl alcohol for 5 min to completely remove the photoresist. Rain-X™ was then applied liberally over the surface of the microwell arrays following the manufacturer's instructions (FIG. 2A, step 4). Deionized water contact angle measurements of APTES regions and Rain-X™-treated regions were obtained using the sessile drop method on a goniometer (DSA100E, KRÜSS GmbH) and fitting the drop shape by the Young-Laplace method (FIG. 2B).

### Coating and Visualization of Capture Antibody in Microwells

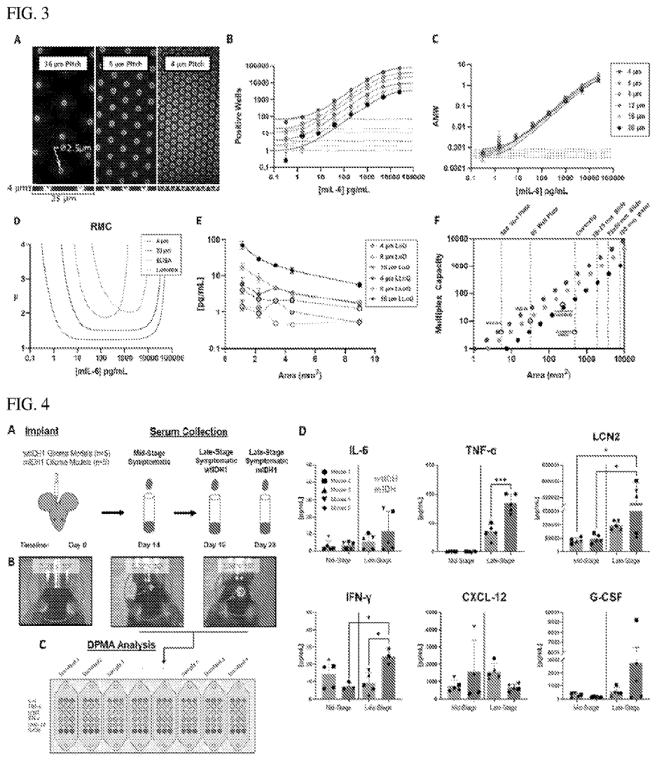

Capture antibodies were dispensed over microwell surfaces using a handheld micropipette and allowed to incubate overnight at room temperature (FIG. 2D, steps 1-2). Unbound capture antibodies were removed by washing DPMA chips with PBS with 0.1% Tween-20 (PBST). To ensure complete removal of unbound capture antibodies, low residue tape was placed over the microwell arrays and then peeled off (FIG. 2D, step 3). Acrylic flow cells were then affixed over microwell arrays with double-sided tape and filled with SuperBlock blocking buffer for 1 h, followed by Casein blocking buffer for an additional hour. To verify the capture antibody coating (FIGS. 2E and F), AF488 goat anti-rat IgG was diluted in PBS, injected into a flow cell, and allowed to incubate for 1 h. The AF488 antibody solution was then washed out of the channel with PBST. Confocal images of the microwells (FIG. 3A) were taken using an oil immersion Plan-Apochromat 100×/1.35 NA objective on an inverted microscope (Olympus IX-81) equipped with an iXON3 EMCCD camera (Andor Technology), OBIS lasers (Coherent) with a USB 6003 data acquisition device (National Instruments), and a Yokogawa CSU-X1 spinning disk confocal. Acquisition of images was controlled by Meta-Morph (Molecular Devices). Z-stack images were captured with 488 nm excitation at exposure time of 200 ms. 3D z-projected images were produced by brightest point projection of z stack image sequences in ImageJ.

### Glioma Mouse Models

### Cell Line and Cell Culture Conditions

The glioma cells [NPA: shp53, NRAS, shATRX (wt-IDH1); NPAI: shp53, NRAS, shATRX, IDH1-R132H (mIDH1)] were developed in house de novo by genetically engineering mouse models and isolating the tumor cells for growth in vitro. These glioma cells also harbor luciferase used for in vivo imaging of the tumor. The method for the glioma cell generation is described previously in detail (Alghamri et al., 2021b). Glioma cells were grown in Dulbecco's Modified Eagle Medium/Nutrient Mixture F-12 (DMEM/F-12) (Gibco, 11320032) supplemented with L-glutamine (Gibco, 25030081), B-27 supplement (Gibco, 17504044), N-2 supplement (Gibco, 17502048), Antibiotic-Antimycotic (Gibco, 15240062), and Normocin (Invivogen, ant-nr-2). Cells were maintained in a humidified incubator at 95% air/5% CO₂ (37° C.) and passaged every 3-5 days.

### Animal Details

Six to eight-week-old female C57BL/6 mice were acquired from Jackson Laboratory. All mice were housed in a pathogen free environment in the University of Michigan vivarium. Tumor implantation was done as described before (Alghamri et al., 2021b). Briefly, mice are anesthetized using ketamine and dexmedetomidine prior to stereotactic implantation with 50,000 glioma cells in the right striatum. The coordinates for implantation are 0.5 mm anterior and 2.0 mm lateral from the bregma and 3.0 mm ventral from the dura. Glioma cells were injected at a rate of 1 L/min. A combination of buprenorphine (0.1 mg/kg) and carprofen (5 mg/kg) was administered for analgesia. Carprofen was administered one day post-implantation as well for analgesia. For serum collection, the glioma mouse models were bled at a mid-stage symptomatic time point (day 13 post implantation) and at the time of euthanasia. The mid-stage symptomatic bleed of 0.1 mL to 0.2 mL was done via submandibular bleed (5 mm Goldenrod animal lancet, MEDIpoint). The late-stage symptomatic bleed was done by decapitation after isoflurane overdose. The blood was left to clot in serum collection tubes (Sarstedt, 50-809-211) for 45 min at room temperature. The blood is then centrifuged at 2000×g for 15 min (4° C.) to isolate the serum. Serum samples were stored at −80° C. Representative images of the glioma progression in vivo were taken using the IVIS Spectrum in vivo imaging system (FIG. 4B) (PerkinElmer, 124262). In brief, 100 μL of luciferin solution was injected 5 min prior to imaging. During the 5 min, mice were anesthetized with oxygen and isoflurane (2.5% isoflurane), followed by loading mice in the IVIS Spectrum device and imaging. On the connected computer, the Living Imaging software (PerkinElmer) was used to select the region of interest (ROI) as a circle over the head, and bioluminescence intensity was measured.

### Digital ELISA Assay

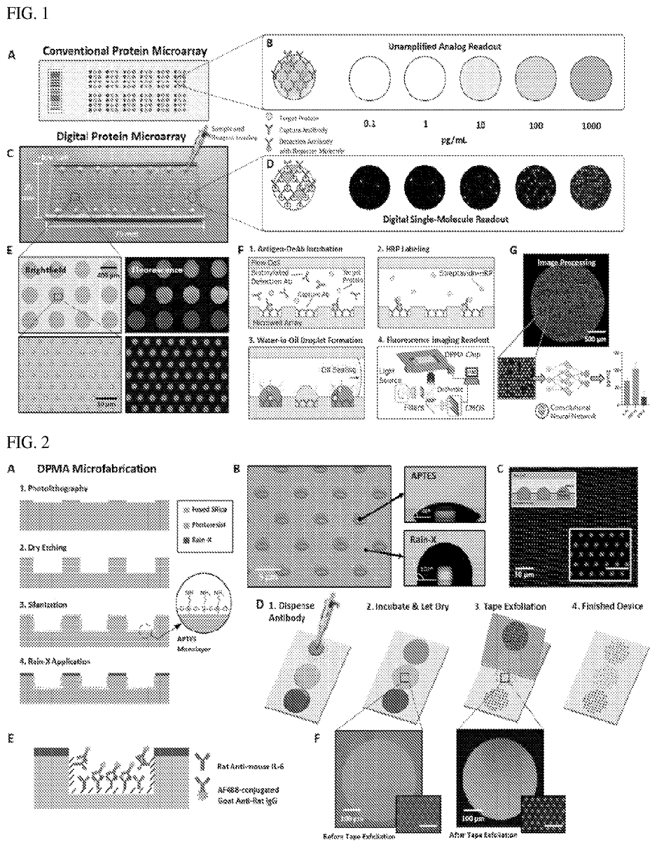

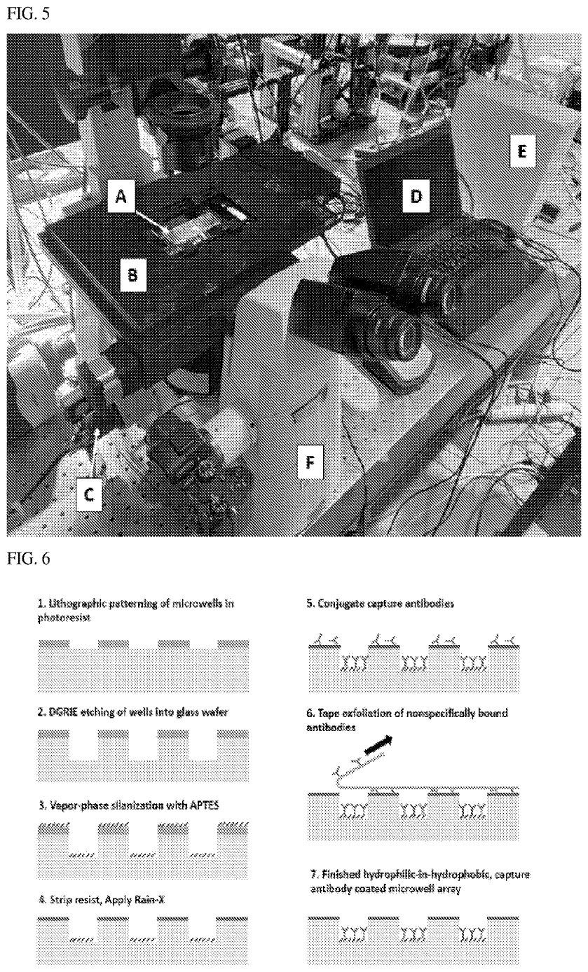

All reagents were prepared in low retention tubes and kept on ice until use. Serum samples and standards were diluted in 50% FBS in Assay Buffer B from Biolegend. Serum samples of 1-10 μL in volume, depending on the dilution factor required, were diluted to a final volume of 30 μL. Standards were created by 5-fold serial dilution in excess volume. Biotinylated detection antibodies were diluted to the working concentration in Assay Buffer B. For multiplex assays, a detection antibody cocktail was created by mixing all detection antibodies together in Assay Buffer B at the required working concentrations. Avidin-HRP was diluted in SuperBlock blocking buffer. Prior to beginning the assay, blocking buffer was washed out of DPMA chips with PBST. For the first assay step, 28 μL of sample or standard was loaded into DPMA chips and mixed in-channel for 15 min (single-plex IL-6, FIG. 3) or 2 h (six-plex assay, FIG. 4) using an automated multichannel pipette system previously described (Song et al., 2021a) to facilitate advective mass transport to the sensor surface. DPMA chips were then washed by flushing the channels with PBST using a separate automated syringe pump for 2 min at 50 μL/min. Using the automated multichannel pipette system again, 28 μL of detection antibody cocktail was loaded onto the DPMA chips, mixed for 10 min, and washed using the same procedure (FIG. 1F, step 1). Next, 40 μL of avidin-HRP solution was slowly loaded into DPMA chips for 1 min, then washed with PBST for 10 min (FIG. 1F, step 2). Finally, 35 μL of QuantaRed substrate solution was manually loaded into DPMA chips and then flushed with 45 μL of HFE-7500 oil (FIG. 1F, step 3). DPMA chips were allowed to incubate for 10 min to ensure sufficient concentration of fluorescent enzyme product within the water-in-oil microdroplets could be achieved for single-molecule counting. DPMA chips were then loaded onto an automated fluorescence microscope imaging system consisting of an inverted epifluorescence microscope (Nikon Eclipse Ti) and programmable X-Y-Z motorized stage (ProScan III, Prior Scientific) (FIG. 1F, step 4) (FIG. 5). The stage was controlled by a computer running a custom LabVIEW program that facilitated autofocusing and imaging of microwell arrays at positions pre-defined in the program (FIG. 5). Microwell array images were collected by a 10× microscope objective (Nikon) and CMOS camera (XIMEA xiC) and saved locally on the computer.

### Image and Data Analysis

Images collected by the imaging system were later analyzed using a previously developed convolutional neural network (CNN) algorithm written in MATLAB (Song et al., 2021c). The CNN was pretrained to recognize and count enzyme active “On” microwells (positive wells) while masking out any defects or contaminations on the microwell array surface. The data produced by the MATLAB-based CNN algorithm was analyzed using GraphPad Prism. Standard curves were fit using the built-in Langmuir adsorption model “One site-Total” and setting the linear nonspecific component to zero. To determine mouse serum biomarker concentrations (FIG. 4), positive wells values were interpolated from a 6-plex standard curve run in parallel on each DPMA device used. Limit of detection (LoD) was calculated as the average background signal plus 3 times the standard deviation (Table 1, “LoD 3×σ” column). Lower limit of quantification (LLoQ) was determined by interpolating the signal corresponding to 100 positive wells (Table 1, “LloQ Poisson” column) or the signal corresponding to the mean plus ten times the background standard deviation (Table 1, “LLoQ 10×σ” column). Resolution of molecular concentration was determined by inputting the standard curve fit parameters calculated by Prism into a Mathematica script made publicly available by Wilson et al. (2022).

### Results and Discussion

### Digital Protein Microarray (DPMA)

FIG. 1 shows the working principle of the Digital Protein Microarray (DPMA) platform. DPMA employs single-molecule counting as a signal amplification strategy to significantly enhance assay sensitivity over conventional protein microarrays. Conventional protein microarrays are manufactured by spotting capture antibodies in a spatially addressable manner on a substrate, commonly at glass slide (FIG. 1A). Samples are then incubated on the substrate in reservoirs, then labeled with a fluorescent or chemiluminescent secondary antibody. The signal is then read out by a microarray scanner and the fluorescence intensity of each capture antibody spot can be used to determine the analyte concentration. The microarray format allows for very high numbers of analyte molecules to be screened while consuming very little sample. In DPMA (FIG. 1C), signal amplification was obtained by splitting capture antibody spots into hundreds of thousands of femtolieter (fL) microwells (FIG. 1E) in which digital ELISA can be carried out. This new assay format for digital ELISA preserves the very high multiplexing capabilities of the traditional protein microarray, with analytical performance exceeding sub-pg/mL.

The assay procedure is similar to previous work (Song et al., 2021a; 2021b; 2021c). To achieve single-molecule ELISA, an acrylic flow cell can be fixed on top of the DPMA (FIG. 1C) to guide samples and reagents over the microwell array sensors. Antigen capture and labeling is performed with conventional sandwich ELISA chemistry (FIG. 1F, Panel 1-2), further details are provided in the FIG. 1 caption and Material and Methods. The sample and detection antibody loading volume (25 μL) was enough to completely displace the volume of the flow cell (13 μL). The washing volume used was sufficient for at least 10× replacement of the flow cell volume which minimized any background signal caused by nonspecific binding. To achieve single-molecule detection, the flow cell is flushed with HRP substrate QuantaRed, then flushed again with HFE oil, forming an array of water-in-oil microdroplets over each well. The fluorogenic enzyme product is confined within each microdroplet, so that sufficient concentration of the fluorophore is maintained to observe the signal from one immune complex (FIG. 1F, Panel 3). Signal readout in DPMA is achieved by epifluorescence microscopy on an automated custom scanner (FIG. 1D, Panel 4). The chip can be imaged using a standard epifluorescence microscope equipped with an automated programmable X-Y-Z stage (FIG. 5). A custom LabVIEW program was developed to automatically drive the chip over the stage and acquire images of each array. This enabled “hands-off” operation and helped control run-to-run imaging consistency. Images of each microwell array collected by the scanner are analyzed using a previously developed AI-enabled MATLAB program (FIG. 1G), (Song et al., 2021c). The MATLAB program counts each well in a binary fashion as “on” or “off” and masks out any defects or contamination on the surface of microwell arrays. Wells containing an analyte molecule appear as bright fluorescence spots in the image, while wells containing no analyte molecule remain dark. Unlike our previous digital immunoassay platform and many digital assays, the DPMA does not use microbeads, so there is no need to include an additional image analysis step to count how many beads are captured in the array. This reduces the scan time and the number of images needed by half and eliminates potential sources of experimental error arising from miscounting beads, wells double-filled with beads, or debris in microwells resulting in bead false-recognition. Since the number of active microwells remains fixed at 100%, the signal measured from each microwell array can simply be reported as the number of positive microwells, or as “average molecules per well (AMW)” which can be calculated by rearranging for 2 in the Poisson distribution (Zhang and Noji, 2017): AMW=λ=. In.1. Ppositive) where Ppositive is the fraction of microwells that are fluorescently active in a particular microwell array. This is analogous to “average enzyme per bead (AEB)”, “average immunocomplex per bead (AIB)”, or similar figures used by other digital immunoassay technologies. The number of positive wells or AMW can be used to calculate the analyte concentration by interpolating from a standard curve.

### Fabrication of DPMA

Specific and high-fidelity patterning of proteins into recessed microwell features at the size scale required for the DPMA device (<10 μm) is not possible with conventional protein patterning methods such as inkjet printing, pin spotting, or dip-pen nanolithography (Bhatt and Shende, 2022). Therefore, a protein patterning method using conventional photolithography and wafer-level microfabrication processes was used (FIG. 2). These processes facilitate both the microwell array manufacturing and enable precise control of the surface chemistry, which is important to maintain compatibility with water-in-oil droplet formation necessary for digital ELISA.

### Microfabrication of Microwell Arrays

Microwell features are first patterned in photoresist on a glass wafer using contact-mode photolithography (FIG. 2A, Step 1). Using photolithography allows for many microwell array designs across a range of sizes, shapes, and densities to be realized. Then, reactive ion etching (RIE) is used to etch the microwells into the glass wafer to a depth of 3.5 μm (FIG. 2A, Step 2) (FIG. 11). After etching, individual DPMA devices containing several microwell arrays were singulated from a wafer using a dicing saw. The entire microfabrication procedure required just one photolithography step and a batch of 8 wafers could be completed within one 8-hour workday by one technician. Two DPMA devices of the size (75×25 mm) used in this work could fit on one 100 mm wafer, so the manufacturing throughput of the microfabrication procedure was 16 DPMA devices per day. DPMA devices could be pre-fabricated in batch and stored until use.

### Patterned Hydrophilic-In-Hydrophobic Surface Chemistry

Protein patterning into microwells in DPMA is enabled by precise control of the substrate surface chemistry. With the photoresist still intact from the RIE process, DPMA devices were treated with O2 plasma to form hydroxyl groups on the inner surface of the well. Then, (3-Aminopropyl)triethoxysilane (APTES) was grafted onto the surface inside the microwells by chemical vapor deposition (CVD) (FIG. 2A, Step 3). The photoresist effectively masks both the O2 plasma hydroxylation process and the silane grafting process, leaving only the microwell surface coated with APTES, while regions outside of the microwells retain their native surface chemistry. After CVD, the photoresist was removed by sonication in acetone and isopropyl alcohol.

Microwell arrays used in digital ELISA should be uniformly hydrophobic on the surface in order facilitate droplet formation during the oil sealing process (Kim et al., 2012), with the optimal configuration being a hydrophilic well recessed in a hydrophobic surface (Tripodi et al., 2018). Materials used for dELISA microwell arrays must also have near-zero autofluorescence to maintain a sufficient signal-to-noise ratio to distinguish “on” from “off” wells. Numerous hydrophobic, low-autofluorescence materials have been demonstrated for bead-based digital ELISA technologies such as PDMS, CYTOP, Teflon, and cyclic olefin copolymer (COC) (Kan et al., 2012, 2020; Kim et al., 2012; Tripodi et al., 2018; Song et al., 2021b). PDMS and COC are incompatible with the silanization process described because they must be molded by soft lithography or injection molding, respectively. CYTOP and Teflon can be micro-machined but they are expensive and challenging to work with in practice. In order to create a hydrophobic surface in DPMA, the automotive glass water repellant Rain-X™ was applied in liquid form over the entire device surface according to the manufacturer's directions (FIG. 2A, Step 4). The hydrophobic component of Rain-X™ is primarily of PDMS, while other additives chemically hydroxylate the surface it is applied to, allowing the PDMS molecules to bind covalently to the surface (Di Justo, 2010). It was found that APTES blocked the Rain-X™ coating process, as areas coated with APTES remained hydrophilic and capable of binding to antibodies while the rest of the surface was made hydrophobic. The water contact angle of APTES-treated regions of DPMA devices was approximately 46° while Rain-X™ regions were approximately 102° (FIG. 2B). This yielded a desirable hydrophilic-in-hydrophobic (HIH) microwell surface configuration and enabled highly uniform water-in-oil microdroplet formation (FIG. 2C).

### Specific Protein Patterning in Microwells

The precisely patterned surface chemistry achieved by the microfabrication process enables highly specific coating of antibodies within the glass microwell arrays. First, a solution of capture antibody in coating buffer is mechanically dispensed onto the microwell array surface and allowed to incubate overnight (FIG. 2D, Step 1-2). Currently this process is performed with a handheld micropipette. A microarray printer can be used to automate this process. Antibodies bind to the amine groups of the APTES molecules and remain permanently fixed (Vashist et al., 2014). DPMA devices are then rinsed in washing buffer and allowed to dry (FIG. 2D, Step 1-2). This initial washing removes most of the nonspecifically bound capture antibody, but a thin layer still remains on the substrate outside of the wells (FIG. 2F). In order to achieve micro-scale resolution of antibody patterning and preserve surface hydrophobicity, the remaining antibodies are removed by “tape exfoliation” (FIG. 2D, Step 3) which comprises applying and quickly peeling off a low residue tape over the entire surface of the DPMA. Finally, an acrylic flow cell is attached with double-sided tape and DPMA devices are then ready for use.

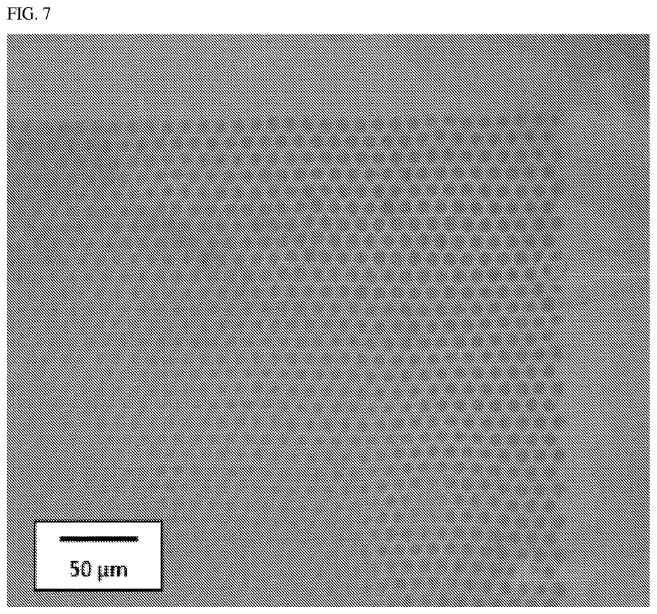

An assay was developed to verify the specificity of the antibody coating (FIGS. 2E and F). First, nonspecific binding sites were blocked with a blocking buffer flushed into DPMA devices. Then, AF488 anti-IgG antibody was injected and allowed to bind to the patterned capture antibodies (FIG. 2E). Excess anti-IgG antibody was flushed out with washing buffer, and any region on the DPMA chip with patterned antibody produced a bright fluorescent signal (FIG. 2F). Complete, specific, and uniform coating of microwells was verified by confocal microscopy (FIG. 3A). Capture antibody could be seen on the sides and bottom of the wells (FIG. 3A, bottom), but not on surface regions between wells. The donut-shaped fluorescence profile of the microwells (FIG. 3A, top) arises from the 3D shape of the well—the sides of the well appear brighter because the entire fluorescence signal from the well sidewalls is projected onto the image plane. This assay was also used to confirm the efficacy of the tape exfoliation method by imaging antibodies that had been removed by the tape after it had been peeled off (FIG. 7).

### Increasing Microwell Density Enables Sensor Miniaturization

In previous bead-based digital ELISA platforms, antibody coated microbeads are settled into microwells by gravity. The fill rate is dependent on the bead concentration and microwell geometry, and varies from device-to-device, increasing the experimental CV. In DPMA, the partition number can be precisely controlled, inconsistencies related to bead loading are eliminated, and the “fill rate” is fixed at 100%. Furthermore, the microwell geometry is no longer constrained to allow optimal bead loading. Leveraging the precision of photolithography, microwell arrays can be designed in any diameter, density, and total area within the process limits. With this new design flexibility, the effect of increasing the partition number per sensor area (microwell density) on the assay performance was evaluated.

### Reduction in Poisson Noise

DPMA chips were fabricated with a range of microwell array densities (FIG. 3A). The microwells were arranged in a hexagonal lattice with the well-to-well pitch (lattice constant) describing the density. In this study, microwell arrays with pitch ranging from 4 to 20 μm were used. The microwell diameter and depth were fixed for each array density at 2.5 μm and 3.5 μm, respectively. Standard curves were generated by serially diluting recombinant mouse IL-6 in 50% fetal bovine serum (FBS) and performing digital ELISA as described in Material and Methods. The data is plotted as the total number of “Positive Wells” and as AMW in FIGS. 3B and C and summarized in Table 1.

It was found that as microwell density is increased, the number of positive wells that are counted for a given concentration also increases. Here, increasing the microwell density is analogous to achieving higher bead filling rate in bead-based dELISA technologies. Generally in digital ELISA, it is advantageous to count as many positive wells as possible in order to minimize the measurement Poisson noise (Kim et al., 2012). It was also found that increasing the microwell density also increased the measurement background. This phenomenon has been reported before, and for other bead-based technologies there has been an optimal number of beads reported for certain high-affinity antibody-antigen pairs (Wu et al., 2022). No such trend was observed over the microwell densities measured and instead it was found that while the background signal continues to increase with density, the limit of detection decreases. Because of the reduction Poisson noise, the higher density microwell arrays also had a lower background coefficient of variation (CV), yielding a lower overall limit of detection. The data indicate that this trend might continue if the microwell pitch were reduced further; however, decreasing microwell pitch places additional demands on the optical system used to image the microwell arrays to resolve individual wells, and higher magnification or numerical aperture of the objective lens is required. Microwell arrays with a 4 μm pitch were the practical limit of the optical system used in this work, and this design was selected for subsequent experiments. No trend was observed between the different microwell densities when the data were analyzed by average molecule per well (AMW). In other words, the spatially averaged “on well” frequency is the same across all microwell densities. This can be explained if one considers the total sensor area as a Langmuir adsorption process. For a given area, there is a certain number of analyte molecules, along with detection antibody-HRP reporter immunocomplexes, that will bind to the surface at equilibrium. Since the curves collapse on each other when spatially averaged, what FIG. 3C shows is that increasing the microwell density only increases the sampling rate at which the molecules on that surface are counted, or the number of partitions in the Poisson process. The background, standard deviation, LoD and LloQ follow the same trend as FIG. 3B. Therefore, either the total number of positive wells or AMW can be used to calculate analyte concentration for the DPMA. The DPMA platform was quantitatively benchmarked against previous platforms and other relevant multiplex immunoassay platforms in Table 3.

### Improvement to Measurement Resolution

Many immunoassay applications require determining, with statistical certainty, a difference between two or more measured samples. While digital immunoassays have received broad attention for achieving extremely low limits of detection (LoD), this metric does not completely describe the assay's analytical capabilities in, for example, monitoring biomarker perturbations over time. Recently, Wilson et al. (2022) have proposed the “resolution of molecular concentration (RMC)” as a new performance metric for immunoassays. RMC can be calculated from curve fitting values and the parameter u indicates the fold difference between two concentrations that can be measured by the assay within a given confidence interval. In the analysis, a confidence interval of 90% was used. In FIG. 3D, the RMC has also been calculated from the commercial ELISA kit from which the mouse IL-6 antibody pairs used in the DPMA fabrication were obtained. Data from a Luminex kit is included as well. The portions of the RMC curves below 3, about 10 to 1000 μg/mL for ELISA and 70 to 8000 μg/mL for Luminex approximately correspond to the manufacturer's rated linear ranges, with vertical asymptotes at the approximate LLOD and ULoQ, respectively. Both the lower-density 20 μm pitch and higher-density 4 μm pitch DPMA devices achieve a lower and wider u value <3 than the analog assays, confirming not only the improvement to sensitivity that single-molecule counting provides, but also a significant improvement to measurement resolution. Beyond improvement to the limit of detection and reduction in Poisson noise, FIG. 3D demonstrates that increasing the microwell density in DPMA, and the digital assay format broadly, also improves the measurement resolution.

### Determination of the Minimum Microwell Array Size

The effect of the total microwell array area (the total number of microwells analyzed) on the limit of detection (LoD) and lower limit of quantification (LLoQ) was next determined. Multiplexing in DPMA is spatially encoded and each microwell array can be coated with a different capture antibody, meaning that the multiplex capacity is ultimately determined by the total microwell array size. Therefore, it is desirable to minimize the microwell array footprint so that the maximum number of sensors can fit into a given substrate area.

For each microwell density examined, the LoD and LLOQ are plotted in FIG. 3E. Limit of detection was defined as 3 standard deviations above the mean background signal and the LLOQ as the concentration providing at least 100 on wells (theoretical Poisson CV<20%), or 10 standard deviations above the mean background signal, whichever was greater. It was found that for all microwell densities, as area was increased both the LoD and LLoQ appear to exponentially decay to lower values. In the data for the 4 μm microwell pitch, one microwell array in the three that were measured had an aberrantly high background signal which increased the CV but was included in analysis. Improvements to LoD and LLOQ appear to be marginal beyond 4 mm², indicating scanning additional area becomes statistically unnecessary depending on the sensitivity required. Generally, the data in FIG. 3E indicate that the optimum sensor performance can be achieved by minimizing the microwell pitch and maximizing the microwell array area, thereby maximizing the total number of wells interrogated in the measurement, which is in good agreement with Poisson statistical theory.

With the LoD and LLoQ determined for each density, FIG. 3F provides the theoretical maximum multiplex capacity for various substate sizes relevant to life sciences applications. The unit area for one sensor was calculated from FIG. 3E as the area with which a given microwell density would yield at least 100 positive wells at 5 μg/mL, which is the lower limit of the manufacturer-rated analytical range of the antibody pairs we used. For the highest microwell density DPMA fabricated, it was determined that approximately 30 could fit in the bottom of a single well of a 96 well plate, for example. This is three times the maximum multiplex capacity of the Mesoscale Diagnostics MULTI-ARRAY platform (10-plex), which is the gold standard technique for sensitive and multiplex detection in 96-well plate format. The DPMA is also not limited by form factor and can readily be fabricated on any substrate size and easily integrated with other microfluidics devices. Recently, an early version of the DPMA for a microfluidic tissue-on-chip study was demonstrated (Su et al., 2023).

### Low-Volume Multiplex Cytokine Detection in Mouse Serum

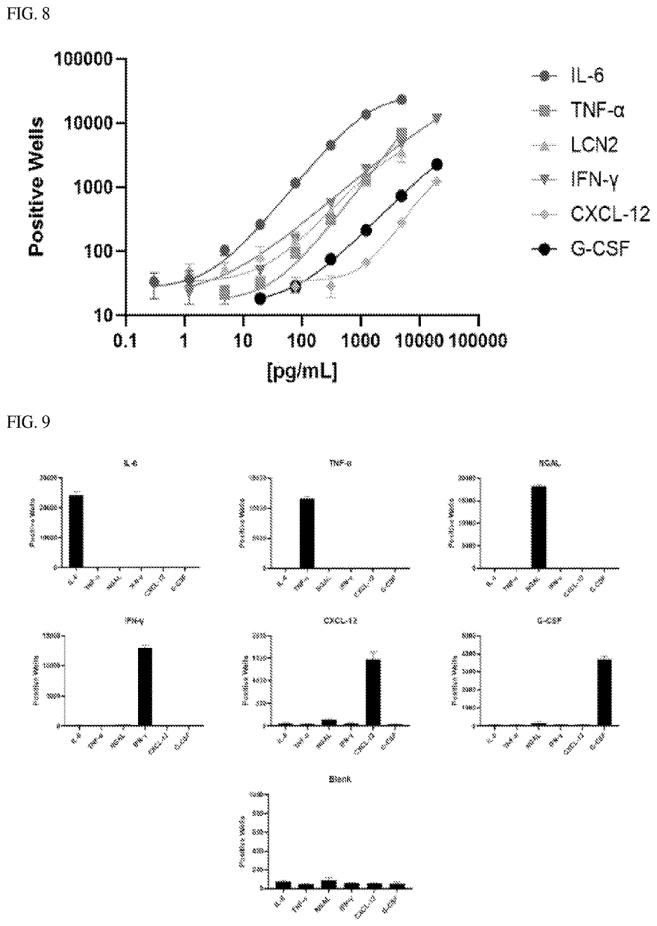

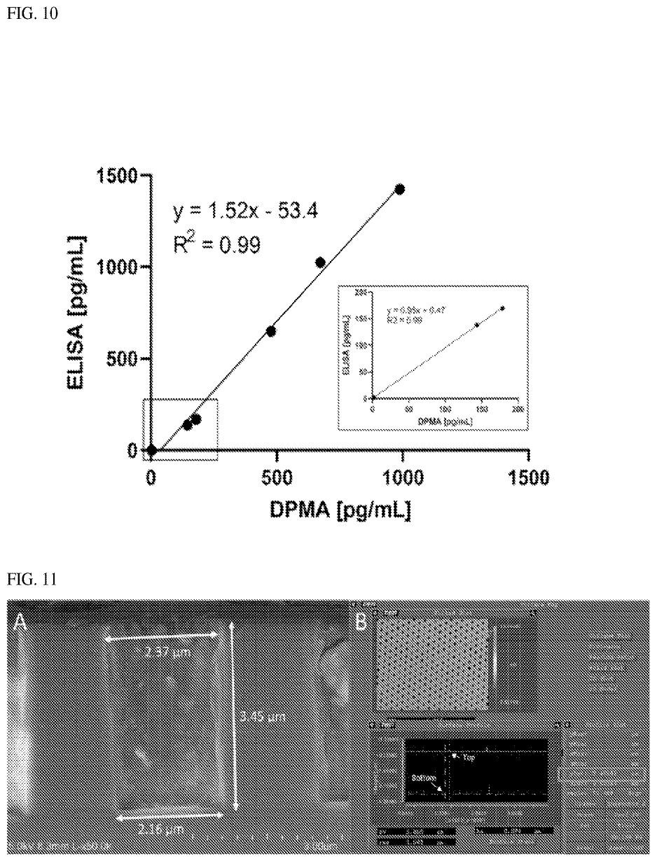

Biomedical research studying the effects of complex processes and diseases, such as aging and cancer, or treatments may involve the use of small animal models, which resemble the biological and behavioral characteristics of larger animals. One downside of small animal models is the limited sample volumes they can provide, restricting the types and number of analyses that can be performed for a single animal. The use of microfluidics in DPMA minimizes the sample volume necessary for a single run, increasing the amount of assays that can be run with a single banked sample. This capability is critical in early-stage research, as it allows for different assay protocols, such as the biomarker panel and dilution factor, to be tested for an application without needing to re-run time consuming and laborious animal experiments. To demonstrate its utility in biomarker discovery and monitoring, we used DPMA to analyze the cytokine levels in the serum of glioma mouse models (FIGS. 4A and B). We developed a six-plex panel consisting of interleukin-6 (IL-6), tumor necrosis factor alpha (TNF-α), interferon-gamma (IFN-γ), lipocalin-2 (LCN2), and C-X-C motif chemokine ligand-12 (CXCL-12) as these molecules had altered secretion in mIDH1 conditioned media (Alghamri et al., 2021b). The standard curve and cross reactivity assessment for the panel is provided in FIGS. 8 and 9. An ELISA correlation is provided for IL-6 in FIG. 10.

Gliomas are tumors of the central nervous system, primarily brain, which are separated into several types based on histopathological, genetic, and epigenetic features (Louis et al., 2021). The mutational status of isocitrate dehydrogenase 1 (IDH1) is used to identify glioma type, as it influences patient clinical outcome by altering the glioma cell biology and the glioma tumor microenvironment (McClellan et al., 2023). Granulocyte colony-stimulating factor (G-CSF), along with several other molecules, was shown to have increased secretion from mutant IDH1 (mIDH1) glioma cells (Alghamri et al., 2021b). Increased systemic G-CSF leads to altered bone marrow granulopoiesis and a reduction of the inhibitory potential of polymorphonuclear myeloid-derived suppressor cells (PMN-MDSCs), which indicates G-CSF as a potential treatment for wildtype IDH1 (wtIDH1) patients (Alghamri et al., 2021b). Given that altered cytokine levels, like that of G-CSF, may lead to changes in the tumor microenvironment and in the body's systemic immune response, here the DPMA was used to quantify levels of several secretory factors in mouse serum of wtIDH1 and mIDH1 glioma harboring mouse models. IL-6, TNF-α, and IFN-γ are proinflammatory cytokines associated with anti-tumoral properties in the glioma tumor microenvironment (Alghamri et al., 2021a). LCN2 is a small protein which supports tumorigenesis by increasing cell proliferation and cancer cell metastatic potential (Hsieh et al., 2021). CXCL-12 is a chemokine associated with the chemotaxis of immunosuppressive MDSCs into the glioma tumor (Alghamri et al., 2022). In the blood, all six of these six secretory factors mentioned above lead to altered immune response systemically and changes in trafficking of immune cells to the tumor. Serum was collected from wtIDH1 and mIDH1 glioma mouse models at mid-stage and late-stage time points (FIG. 4A). Similar to that of wildtype and mutant IDH1 harboring glioma patients, mIDH1 glioma mouse models survive longer (median survival: 33 days) than the wtIDH1 glioma mouse models (median survival: 21 days) (Alghamri et al., 2021b), so the late-stage blood was collected from the wtIDH1 and mIDH1 glioma mouse models on days 19 and 28 respectively. Here, it was observed that late-stage G-CSF in the mIDH1 glioma mouse models' serum is increased compared to that from the wtIDH1 glioma mouse models (FIG. 4D), which is predicted based on previous findings (Alghamri et al., 2021b). The mutation of IDH1 by the glioma cells also correlates with increased TNF-α, increased LCN2, and decreased CXCL12 in the serum of late-stage glioma mouse models (FIG. 4D). These results implicate the mutation of IDH1 in the glioma cells affect the immune response systemically by inducing different migration patterns of MDSCs and increasing the stimulation of immune cells via CXCL12 and TNF-α, respectively. The increased LCN2 is one mechanism, by which, mIDH1 may support tumor progression. No changes in the mid-stage time point are observed between the wtIDH1 and mIDH1 glioma mouse model serum, indicating that the mIDH1-induced changes in serum CXCL12 and TNF-α levels happens later in glioma progression and is thus dependent on tumor burden.

Provided herein is a compact and sensitive microarray-format multiplexed immunoassay that achieves signal amplification by single-molecule counting. Such a platform is urgently needed for biomarker validation, which is trending quickly toward panels of low-abundance biomarkers correlated to disease states. The simple spatial multiplex encoding strategy and sensor miniaturization of the platform was enabled by photolithographic patterning of proteins within microwell arrays.

TABLE 1
Background signal, LoD, and LLoQ of microwell pitch analyzed by positive wells or AMW.

| | Positive Wells | | | | | | Average Molecules Per Well (AMW) | | | | | |
|---|---|---|---|---|---|---|---|---|---|---|---|---|
| | | | | LoD | LLoQ | LLoQ | | | | LoD | LLoQ | LLoQ |
| Microwell | | | | 3 × [symbol] | Po [symbol] | [symbol] 0 × [symbol] | | | | 3 × [symbol] | Po [symbol] | 16 × [symbol] |
| Pitch | Background | [symbol] | CV | (pg/mL) | (pg/mL) | (pg/mL) | Background | [symbol] | CV | (pg/mL) | (pg/mL) | (pg/mL) |
| 4 μm | 69.2 | 4.32 | 0. [symbol] | 0.61 | 1.77 | 2.4 [symbol] | 8.28 [symbol] 10 [symbol] | [symbol] .20 × | 0.06 | 0.69 | 2.01 | 2.82 |
| | | | | | | | | 10 [symbol] | | | | |
| 6 μm | 19.5 | 4.96 | 0. [symbol] | 0.78 | 8.92 | 5.49 | [symbol] .28 × 10 [symbol] | 1.32 × | 0.2 [symbol] | 0. [symbol] | 10.42 | 6.41 |
| | | | | | | | | 10 [symbol] | | | | |
| 8 μm | 12.2 | 4.2 [symbol] | 0.35 | 0.84 | 16.1 | 7.7 | 5.99 × 10 [symbol] | 2.07 × | 0.3 [symbol] | 1.04 | 20.07 | 9.59 |
| | | | | | | | | 10 [symbol] | | | | |
| 12 μm | 3.63 | 1.92 | 0.53 | 2.65 | 38.7 | 7. [symbol] | 3.92 × 10 [symbol] | 2.08 × | 0. [symbol] | 3.0 [symbol] | 44. [symbol] 1 | 8.74 |
| | | | | | | | | 10 [symbol] | | | | |
| 16 μm | 1.88 | 1.3 [symbol] | 0.7 [symbol] | 3.59 | 94.95 | 12.93 | 3.59 × 10 [symbol] | 2.60 × | 0.72 | 4.29 | 112.9 [symbol] | 15. [symbol] 2 |
| | | | | | | | | 10 [symbol] | | | | |
| 20 μm | 1.00 | 0.76 | 0.76 | 5.05 | 209.71 | 15.61 | [symbol] .02 × 10 [symbol] | 2.28 × | 0.7 [symbol] | 5.76 | 237.58 | 17.77 |
| | | | | | | | | 10 [symbol] | | | | |

[symbol]

indicates data missing or illegible when filed

TABLE 2
Analytical details of the 6-plex mouse glioma biomarker panel. Limit of detection is
calculated as 3 standard deviations above the background signal. The analytical range is the
lowest standard concentration above the LoD to the highest standard concentration used to
generate a standard curve as shown in FIG. 8.

| | DPMA 6-Plex | DPMA 6-Plex | Antibody Pair | |
|---|---|---|---|---|
| Analyte | LoD (pg/mL) | Analytical Range | Manufacturer | Cat. No. |
| IL-6 | 1.36 | 4.88-1250 | BioLegend | 504501, 504601 |
| TNF-α | 29.76 | 31.75-5000 | BioLegend | 510802, 506312 |
| LCN2 | 19.81 | 78.13-5000 | R&D Systems | DY1857 |
| IFN-γ | 3.03 | 4.88-20000 | R&D Systems | DY485 |
| CXCL-12 | 696.19 | 1250-20000 | R&D Systems | DY460 |
| G-CSF | 119.73 | 312.5-20000 | R&D Systems | DY414 |

TABLE 3
DPMA cost and performance benchmarking.

| | Instrument | | | LoD* | Sample Volume* | |
|---|---|---|---|---|---|---|
| Diagnostic System | Cost | Assay Time | | (pg/mL) | (μL) | Plexity |
| Colorimetric ELISA | $5,000 | >4 | hours | 1-10 | 100 | 1 |
| Laminex | >$40,000 | >4 | hours | 0.1-100 | 25-50 | 25-65 |
| Quanterix SIMOA | >$200,000 | 60 | min | 0.002-1 | 100 | 6 |
| | >$50,000 | >1.5 | hours | 0.2-1 | 25 | |
| PEdELISA | <$5,000 | <30 | min | 0.1-3 | 15 | 16 |
| (previous platform) | | | | | | |
| DPMA (current platform) | <$5,000 | 30*-160 | min | 0.5-3 | 10 | 6 (demonstrated) |
| | | | | | | 48 (possible)** |

*For high-affinity antibody pairs, such as those for IL-6
**The multiplex capacity for the device shown is 48 with 4 technical replicates for each biomarker. Each circular microwell array contains sufficient number of microwells for one measurement, each row contains four microwell arrays targeting one biomarker (i.e. red-labeled antibody for IL-6, green labeled antibody for TNF-α, blue-labeled antibody for IFN-γ). Each row of four microwell arrays could be functionalized with a different antibody. In each flow cell, the DPMA device shown can fit 48 such rows and each flow cell can hold one sample.

### REFERENCES

Alghamri, M. S., Mcclellan, B. L., Hartlage, C. S., Haase, S., Faisal, S. M., Thalla, R., Dabaja, A., Banerjee, K., Carney, S. V., Mujeeb, A. A., Olin, M. R., Moon, J. J., Schwendeman, A., Lowenstein, P. R., Castro, M. G., 2021a. Targeting neuroinflammation in brain cancer: uncovering mechanisms, pharmacological targets, and neuropharmaceutical developments. Front. Pharmacol. 12, 680021.
Alghamri, M. S., Mcclellan, B. L., Avvari, R. P., Thalla, R., Carney, S., Hartlage, C. S., Haase, S., Ventosa, M., Taher, A., Kamran, N., Zhang, L., Faisal, S. M., Núnez, F. J., Garcia-Fabiani, M. B., Al-Holou, W. N., Orringer, D., Hervey-Jumper, S., Heth, J., Patil, P. G., Eddy, K., Merajver, S. D., Ulintz, P. J., Welch, J., Gao, C., Liu, J., Núnez, G., Hambardzumyan, D., Lowenstein, P. R., Castro, M. G., 2021b. G-CSF secreted by mutant IDH1 glioma stem cells abolishes myeloid cell immunosuppression and enhances the efficacy of immunotherapy. Sci. Adv. 7 (40), eabh3243. Alghamri, M. S., Banerjee, K., Mujeeb, A. A., Mauser, A., Taher, A., Thalla, R., Mcclellan, B. L., Varela, M. L., Stamatovic, S. M., Martinez-Revollar, G., Andjelkovic, A. V., Gregory, J. V., Kadiyala, P., Calinescu, A., Jimenez, J. A., Apfelbaum, A. A., Lawlor, E. R., Carney, S., Comba, A., Faisal, S. M., Barissi, M., Edwards, M. B., Appelman, H., Sun, Y., Gan, J., Ackermann, R., Schwendeman, A., Candolfi, M., Olin, M. R., Lahann, J., Lowenstein, P. R., Castro, M. G., 2022. Systemic delivery of an adjuvant CXCR4-CXCL12 signaling inhibitor encapsulated in synthetic protein nanoparticles for glioma immunotherapy. ACS Nano 16 (6), 8729-8750.
Bhatt, M., Shende, P., 2022. Surface patterning techniques for proteins on nano- and micro-systems: a modulated aspect in hierarchical structures. J. Mater. Chem. B 10 (8), 1176-1195.
Borrebaeck, C., 2018. Precision diagnostics: moving towards protein biomarker signatures of clinical utility in cancer. Nat. Rev. Cancer 17, 199-204.
Cohen, L., Walt, D. R., 2019. Highly sensitive and multiplexed protein measurements. Chem. Rev. 119 (1), 293-321.
Di Justo, P., 2010. What's inside: rain-X. Better driving through alcohol. Wired. https://www.wired.com/2010/06/st-whatsinside-rainx/.
Duffy, D. C., 2023. Digital detection of proteins. Lab Chip 23 (5), 818-847.
Ellington, A. A., Kullo, I. J., Bailey, K. R., Klee, G. G., 2010. Antibody-based protein multiplex platforms: technical and operational challenges. Clin. Chem. 56 (2), 186-193.
Hsieh, Y. H., Tsai, J. P., Yu, C. L., Lee, C. C., Hsu, J. C., Chen, J. C., 2021. Overexpression of lipocalin-2 inhibits proliferation and invasiveness of human glioblastoma multiforme cells by activating ERK targeting cathepsin D expression. Biology 10 (5), 390.
Kan, C. W., Rivnak, A. J., Campbell, T. G., Piech, T., Rissin, D. M., Mosl, M., Peterça, A., Niederberger, H. P., Minnehan, K. A., Patel, P. P., Ferrell, E. P., Meyer, R. E., Chang, L., Wilson, D. H., Fournier, D. R., Duffy, D. C., 2012. Isolation and detection of single molecules on paramagnetic beads using sequential fluid flows in microfabricated polymer array assemblies. Lab Chip 12 (5), 977-985.
Kan, C. W., Tobos, C. I., Rissin, D. M., Wiener, A. D., Meyer, R. E., Svancara, D. M., Comperchio, A., Warwick, C., Millington, R., Collier, N., Duffy, D. C., 2020. Digital enzyme-linked immunosorbent assays with sub-attomolar detection limits based on low numbers of capture beads combined with high efficiency bead analysis. Lab Chip 20, 2122-2135.
Kim, S. H., Iwai, S., Araki, S., Sakakihara, S., lino, R., Noji, H., 2012. Large-scale femtoliter droplet array for digital counting of single biomolecules. Lab Chip 12 (23), 4986-4991. Louis, D. N., Perry, A., Wesseling, P., Brat, D. J., Cree, I. A., Figarella-Branger, D., Hawkins, C., Ng, H. K., Pfister, S. M., Reifenberger, G., Soffietti, R., von Deimling, A., Ellison, D. W., 2021.
The 2021 WHO classification of tumors of the central nervous system: a summary. Neuro Oncol. 23 (8), 1231-1251.
McClellan, B. L., Haase, S., Nunez, F. J., Alghamri, M. S., Dabaja, A. A., Lowenstein, P. R., Castro, M. G., 2023. Impact of epigenetic reprogramming on antitumor immune responses in glioma. J. Clin. Invest. 133 (2), e163450.
Park, K. C., Gaze, D. C., Collinson, P. O., Marber, M. S., 2017. Cardiac troponins: from myocardial infarction to chronic disease. Cardiovasc. Res. 113 (14), 1708-1718.
Ren, A. H., Diamandis, E. P., Kulasingam, V., 2021. Uncovering the depths of the human proteome: antibody-based technologies for ultrasensitive multiplexed protein detection and quantification. Mol. Cell. Proteomics 20, 100155.
Rifai, N., Gillette, M., Carr, S., 2006. Protein biomarker discovery and validation: the long and uncertain path to clinical utility. Nat. Biotechnol. 24, 971-983.
Rissin, D. M., Kan, C. W., Campbell, T. G., Howes, S. C., Fournier, D. R., Song, L., Piech, T., Patel, P. P., Chang, L., Rivnak, A. J., Ferrell, E. P., Randall, J. D., Provuncher, G. K., Walt, D. R., Duffy, D. C., 2010. Single-molecule enzyme-linked immunosorbent assay detects serum proteins at subfemtomolar concentrations. Nat. Biotechnol. 28 (6), 595-599.
Rissin, D. M., Kan, C. W., Song, L., Rivnak, A. J., Fishburn, M. W., Shao, Q., Piech, T., Ferrell, E. P., Meyer, R. E., Campbell, T. G., Fournier, D. R., Duffy, D. C., 2013. Multiplexed single molecule immunoassays. Lab Chip 13, 2902-2911.
Sarma, A., Calfee, C. S., Ware, L. B., 2020. Biomarkers and precision medicine: state of the art. Crit. Care Clin. 36 (1), 155-165.
Song, Y., Ye, Y., Su, S. H., Stephens, A., Cai, T., Chung, M. T., Han, M. K., Newstead, M. W., Yessayan, L., Frame, D., Humes, H. D., Singer, B. H., Kurabayashi, K., 2021a. A digital protein microarray for COVID-19 cytokine storm monitoring. Lab Chip 21 (2), 331-343.
Song, Y., Sandford, E., Tian, Y., Yin, Q., Kozminski, A. G., Su, S. H., Cai, T., Ye, Y., Chung, M. T., Lindstrom, R., Goicochea, A., Barabas, J., Olesnavich, M., Rozwadowski, M., Li, Y., Alam, H. B., Singer, B. H., Ghosh, M., Choi, S. W., Tewari, M., Kurabayashi, K., 2021b. Rapid single-molecule digital detection of protein biomarkers for continuous monitoring of systemic immune disorders. Blood 137 (12), 1591-1602.
Song, Y., Zhao, J., Cai, T., Stephens, A., Su, S. H., Sandford, E., Flora, C., Singer, B. H., Ghosh, M., Choi, S. W., Tewari, M., Kurabayashi, K., 2021c. Machine learning-based cytokine microarray digital immunoassay analysis. Biosens. Bioelectron. 180, 113088.
Su, S. H., Song, Y., Stephens, A., Situ, M., McCloskey, M. C., McGrath, J. L., Andjelkovic, A. V., Singer, B. H., Kurabayashi, K., 2023. A tissue chip with integrated digital immunosensors: in situ brain endothelial barrier cytokine secretion monitoring. Biosens. Bioelectron. 224, 115030.
Tighe, P., Negm, O., Todd, I., Fairclough, L., 2013. Utility, reliability and reproducibility of immunoassay multiplex kits. Methods 61 (1), 23-29.
Tripodi, L., Ven, K., Kil, D., Rutten, I., Puers, R., Lammertyn, J., 2018. Teflon-on-Glass molding enables high-throughput fabrication of hydrophilic-in-hydrophobic microwells for bead-based digital bioassays. Materials 11, 2154.
Vashist, S. K., Lam, E., Hrapovic, S., Male, K. B., Luong, J. H., 2014. Immobilization of antibodies and enzymes on 3-aminopropyltriethoxysilane-functionalized bioanalytical platforms for biosensors and diagnostics. Chem. Rev. 114 (21), 11083-11130.
Waury, K., Willemse, E. A. J., Vanmechelen, E., Zetterberg, H., Teunissen, C. E., Abeln, S., 2022. Bioinformatics tools and data resources for assay development of fluid protein biomarkers. Biomark. Res. 83 (10).
Wilson, B. D., Eisenstein, M., Soh, H. T., 2022. Comparing assays via the resolution of molecular concentration. Nat. Biomed. Eng. 6 (3), 227-231.
Witters, D., Knez, K., Ceyssens, F., Puers, R., Lammertyn, J., 2013. Digital microfluidics-enabled single-molecule detection by printing and sealing single magnetic beads in femtoliter droplets. Lab Chip 13, 2047-2054.
Wu, C., Dougan, T. J., Walt, D. R., 2022. High-throughput, high-multiplex digital protein detection with attomolar sensitivity. ACS Nano 16 (1), 1025-1035.
Zandi Shafagh, R., Decrop, D., Ven, K., Vanderbeke, A., Hanusa, R., Breukers, J., Pardon, G., Haraldsson, T., Lammertyn, J., van der Wijngaart, W., 2019. Reaction injection molding of hydrophilic-in-hydrophobic femtolitre-well arrays. Microsyst. Nanoeng. 5, 25.
Zetterberg, H., Blennow, K., 2020. Blood biomarkers: democratizing alzheimer's diagnostics. Neuron 106 (6), 881-883.
Zhang, Y., Noji, H., 2017. Digital bioassays: theory, applications, and perspectives. Anal. Chem. 89 (1), 92-101.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1. A method of arraying biological molecules on a substrate, comprising:
a) patterning said substrate with protein-binding silane spots;
b) applying an antibiofouling coating to said substrate;
c) coating said substate with said biological molecules; and
d) contacting said substrate with an exfoliation material to remove nonsilane-bound protein.

2. The method of claim 1, wherein said substrate is a surface.

3. The method of claim 1, wherein said substrate or surface comprises a material selected from the group consisting of glass, plastic, and metal.

4. The method of claim 1, wherein said substrate is selected from the group consisting of a well in a microtiter plate, a film, a microscope slide, and a bead.

5. The method of claim 1, wherein said substrate comprises lithographically patterned microwells.

6. The method of claim 1, wherein said exfoliation material is tape.

7. The method of claim 6, wherein said tape is a low residue tape.

8. The method of claim 7, wherein said tape is low-residue semiconductor grade tape or cellophane tape.

9. The method of claim 1, wherein said antibiofouling coating comprising polysiloxanes.

10. The method of claim 9, wherein said polysiloxanes comprise hydroxy-terminated polydimethylsiloxane.

11. The method of claim 1, wherein said biological molecules are selected from the group consisting of proteins, nucleic acid, lipids, and carbohydrates.

12. The method of claim 11, wherein said proteins are antibodies.

13. The method of claim 1, wherein each of said spots comprises one or more distinct biological molecules.

14. The method of claim 1, wherein said array comprises the same or different biological molecules.

15. The method of claim 1, wherein each of said spots is less than 2 μm in diameter.

16. The method of claim 1, wherein each of said spots is less than 1 nm in diameter.

17. The method of claim 1, wherein said biological molecules are only present in said spots.

18. A substrate comprising an array of biological molecules manufactured by the method of claim 1.

19. A method of performing an assay, comprising:
a) contacting the substrate of claim 18 with a test sample; and
b) measuring one or more properties of said biological molecules in the presence and absence of said test sample.

20. The method of claim 18, wherein said assay measures the presence of an analyte in said test sample.

21-27. (canceled)
